(19) United States
(12) Patent Application Publication
(10) Pub. No.: US 2015/0094690 A1
(43) Pub. Date: Apr. 2, 2015

(54) GUIDEWIRE WITH VARYING PROPERTIES

(71) Applicant: Abbott Cardiovasular Systems Inc., Santa Clara, CA (US)

(72) Inventors: John A. Simpson, Carlsbad, CA (US); Raleigh A. Purtzer, Temecula, CA (US); Matthew J. Gillick, Murrieta, CA (US)

(73) Assignee: Abbott Cardiovasular Systems Inc., Santa Clara, CA (US)

(21) Appl. No.: 14/042,321

(22) Filed: Sep. 30, 2013

(51) Int. Cl. A61M 25/09; B21C 3/02

(52) U.S. Cl. CPC A61M 25/09; B21C 3/02; A61M 2025/09108; A61M 2025/09133
USPC 604/528; 72/377; 72/276

(57) ABSTRACT

A method of making a core metal element for a medical guidewire comprising providing a wire of nickel titanium alloy having a length that includes a proximal portion having a first diameter and a distal portion having a second diameter. Applying cold work to the distal portion and not applying cold work to the proximal portion, thereby imparting to the distal portion a third diameter that is smaller than the second diameter; and then applying a reducing process to the wire whereby the proximal portion is reduced to have a fourth diameter that is less than the first diameter.

### BACKGROUND

The present application relates to guidewires configured for intraluminal application in medical procedures, and methods of their manufacture. More specifically, the application relates to guidewires that possess varying properties of flexibility and torsional stiffness along their length.

Guidewires have long been known and used in the art of minimally invasive medical practice. Guidewires are typically used in conjunction with catheters in a procedure under which a placement catheter may first be threaded into the vasculature of a patient to a desired location using known techniques. A lumen within the placement catheter permits the physician to insert a guidewire through the catheter to the same location. Thereafter, when the physician may need to sequentially place a second, or third, or even a fourth catheter to the same location, it is a simple matter to withdraw the catheter while leaving the guidewire in place. After this action, second, third, and fourth etc. catheters may be sequentially introduced and withdrawn over the guidewire that was left in place. In other techniques, a guidewire may be introduced into the vasculature of a patient without the assistance of a placement catheter, and once in position, catheters may be sequentially inserted over the guidewire as desired.

It is typical that best medical practice for anatomical insertion requires a guidewire that has behavioral characteristics that vary along its length. For example, under some conditions, the distal end of the guidewire may be required to be more flexible than the proximal end so that the distal end may more easily be threaded around the more tortuous distal branches of the luminal anatomy. Further, the proximal end of the guidewire may be required to have greater torsional stiffness than the distal end because, upon rotation of the guidewire, the proximal end must carry all the torsional forces that are transmitted down the length of the guidewire from the distal end, whereas the distal end must transmit only those torsional forces that are imparted locally.

Finally, the distal end of a guidewire should be selectively formable, so that the treating physician may apply a curve to the tip of the catheter in order to facilitate navigation along the tortuous passageways of the vascular anatomy. By selectively formable, it is meant that the wire from which the guidewire core is made may be bent to a particular shape and that the shape will be maintained by the wire. This allows the physician to impart a particular shape to the guidewire, by bending or kinking it for example, to facilitate steering its placement into a patient's vasculature. To provide this selective formability, in typical embodiments, the entire core wire may be made of stainless steel. However, other materials may be used to provide this feature. The use of a formable material, such as stainless steel, provides advantages in the guide wire over materials that cannot be formed, such as superelastic materials like Nitinol. Superelastic materials like Nitinol are so resilient that they tend to spring back to their original shape even if bent, thus are not formable. Although superelastic material may be provided with a “preformed” memory shape, such a preformed shape is typically determined in the manufacture of the guide wire and cannot readily be altered or modified by the physician by simply bending the guide wire prior to use. Although use of superelastic materials such as Nitinol in guide wire applications may provide some advantages in certain uses, a formable core, such as of stainless steel, which can be formed by the physician to a shape suitable for a particular patient or preferred by that physician, provides an advantage that cannot be obtained with a superelastic core guide wire.

Thus, certain solutions have been developed in the prior art to address these requirements. In one typical solution, a guidewire may be fabricated by applying the same metallurgical process along the entire length of an initial ingot of uniform metallurgical properties and uniform diameter that will be converted into the guidewire. The initial ingot may be taken up and cold worked along its entire length, or annealed, or swaged, or whatever process is required to impart the desired characteristics to the metal of the final guidewire product. Once these metallurgical processes have been performed on the wire as a whole, the wire obtained from the worked ingot may be geometrically shaped in order to impart desired different flexibilities, torsional stiffnesses and the like that are desired in the final guidewire product. For example, a worked ingot may be shaped by known process such as chemical washes, polishes, grinding, or compressing, to have a distal end with a diameter that is smaller than the diameter of the proximal end. By this means, the distal end will be given greater flexibility but less torsional resistance than the proximal end. A shaped guidewire 10 of the kind described is depicted in FIG. 1 where it may be seen that a core metal element 12 having a configuration with varying diameter sizes along its length is coated in a polymer 14, or other suitable material to add lubricity. The coating may be configured to impart a uniform outside diameter to the overall guidewire 10.

In another typical solution, different pieces of wire may be formed by different processes to have different properties. These pieces of wire may then be joined or connected together into a single guidewire core using known jointing processes, to provide a resulting guidewire with varying properties along its length. For example, as may be envisaged with reference to FIG. 5 through FIG. 9, different embodiments 20a, 20b, and 20c show how a superelastic portion of wire 22a, 22b, and 22c made from Nitinol or similar metal, may be joined to a portion of wire 24a, 24b, and 24c that has linear elastic properties using jointing methods such as welding, or covering with a jacket 26b, or inserting a filler 28c.

Thus, in a core wire having this combination of a distinct and joined formable distal portion and a superelastic proximal portion, desired shapes may be imparted by a physician to the distal end of the guide wire to facilitate making turns, etc., in tortuous vessel passages, while in the same guide wire the more proximal portion would possess superelastic properties to allow it to follow the distal portion through the tortuous passages without permanently deforming.

However, problems may arise in the prior art as described. Welds are generally undesirable on a guidewire because they introduce a potential point of kinking or fracture. Furthermore, discrete steps in the gradient of a guidewire diameter that are introduced by grinding or other known means may also introduce potential points at which stress is raised to produce cracking or fracture.

Thus there is a need in the art for a system and method for a guidewire that solves the problems in the prior art. The present invention addresses these and other needs.

### SUMMARY OF THE INVENTION

In some preferred embodiments, the invention is a method for making a core metal element for a medical guidewire. The method comprises providing a wire of nickel titanium alloy having a length that includes a proximal portion having a first diameter and a distal portion having a second diameter. In some embodiments, the first diameter may be the same as the second diameter. Once a suitable length of wire is selected, cold work is applied to the distal portion, while no cold work is applied to the proximal portion. By this action, there is imparted to the distal portion a third diameter that is smaller than the second diameter. In other words, the diameter of the distal portion is slightly diminished by the application of cold work. Thereafter, a reducing process is applied to the wire whereby the proximal portion is reduced to have a fourth diameter that is less than the first diameter. By this process, the reducing process may diminish the larger diameter of the proximal portion. The reducing process may stop when the diameters of the proximal portion and the distal portion are initially the same, or, in other words, when the fourth diameter is the same as the third diameter. Or, the reducing process may continue to diminish the diameters of both the proximal and the distal portions, such that they each have a fifth diameter that is smaller than the third diameter.

In preferred embodiments, the step of providing a wire includes providing a wire with superelastic properties throughout the length, and the step of applying cold work to the distal portion includes applying sufficient cold work to render the distal portion to have linear elastic properties. By imparting linear elastic properties to the distal portion, that portion becomes formable by the physician. Furthermore, after applying cold work to the distal portion, the proximal portion retains its original superelastic properties as no cold work has been applied to that portion. Notably, no welding process is applied to the wire over the length, and no joint is created or inserted into the wire over the length.

In some embodiments, the step of applying a reducing process to the guidewire includes applying centerless grinding. In other embodiments the step of applying a reducing process includes chemical wash or electrochemical removal, or an electrochemical or mechanical a polishing process.

In some embodiments the step of applying cold work to the distal portion includes drawing the distal portion through a die, and in further embodiments the guidewire may be removed from the die without drawing the distal portion back through the die. In other embodiments, the step of applying cold work to the distal portion includes applying cold work methods selected from: swaging, tensioning, rolling, stamping, and coining.

In some embodiments, the step of providing a wire includes providing a wire wherein the proximal portion is adjacent the distal portion.

In some embodiments, the step of providing a wire includes providing a wire wherein the proximal portion is adjacent a proximal end of the wire, or, wherein the distal portion is adjacent a distal end of the wire.

In some embodiments, the invention is a medical guidewire comprising a solid metal core having a length and having a constant diameter over the length, wherein the length includes a proximal portion having pseudoelastic properties and a distal portion having linear elastic properties. The length of the core does not include a mechanical joint at any location situated between the proximal portion and the distal portion. The length of the core also does not include a metallurgical joint, such as a solder, braze, or weld joint, at any location situated between the proximal portion and the distal portion. In further embodiments, the proximal portion is formed from a nickel titanium alloy, and in yet further embodiments, the distal portion includes metal to which the linear elastic properties have been imparted by a process of cold working.

These, and further advantages of the invention will become apparent when read in conjunction with the figures and the detailed description of the preferred embodiments.

### BRIEF DESCRIPTION OF THE DRAWINGS

FIG. 1 shows a partial sectional view of a prior art guidewire with a sequence of diameter reductions, shown in shortened schematic form.

FIG. 2 is a sectional view through the guidewire of FIG. 1, taken substantially along the line 2-2 in FIG. 1.

FIG. 3 is a sectional view through the guidewire of FIG. 1, taken substantially along the line 3-3 in FIG. 1.

FIG. 4 is a sectional view through the guidewire of FIG. 1, taken substantially along the line 4-4 in FIG. 1.

FIG. 5 shows a sectional view of a prior art guidewire with proximal and distal portions joined together.

FIG. 6 is a sectional view through the guidewire of FIG. 5, taken substantially along the line 6-6 in FIG. 5.

FIG. 7 shows a sectional view of a prior art guidewire with proximal and distal portions joined together.

FIG. 8 is a sectional view through the guidewire of FIG. 7, taken substantially along the line 8-8 in FIG. 7.

FIG. 9 shows a sectional view of a prior art guidewire with proximal and distal portions joined together.

FIG. 10 is a schematic side view of a wire in a first condition in the process of preparation for use according to an embodiment of the present invention.

FIG. 11 is a schematic side view of a wire in a second condition in the process of preparation for use according to an embodiment of the present invention.

FIG. 12 is a schematic side view of a wire in a third condition in the process of preparation for use according to an embodiment of the present invention.

FIG. 13 is a schematic side view of a wire in a fourth condition in the process of preparation for use according to an embodiment of the present invention.

FIG. 14 is a schematic side view of a wire in a fifth condition in the process of preparation for use according to an embodiment of the present invention.

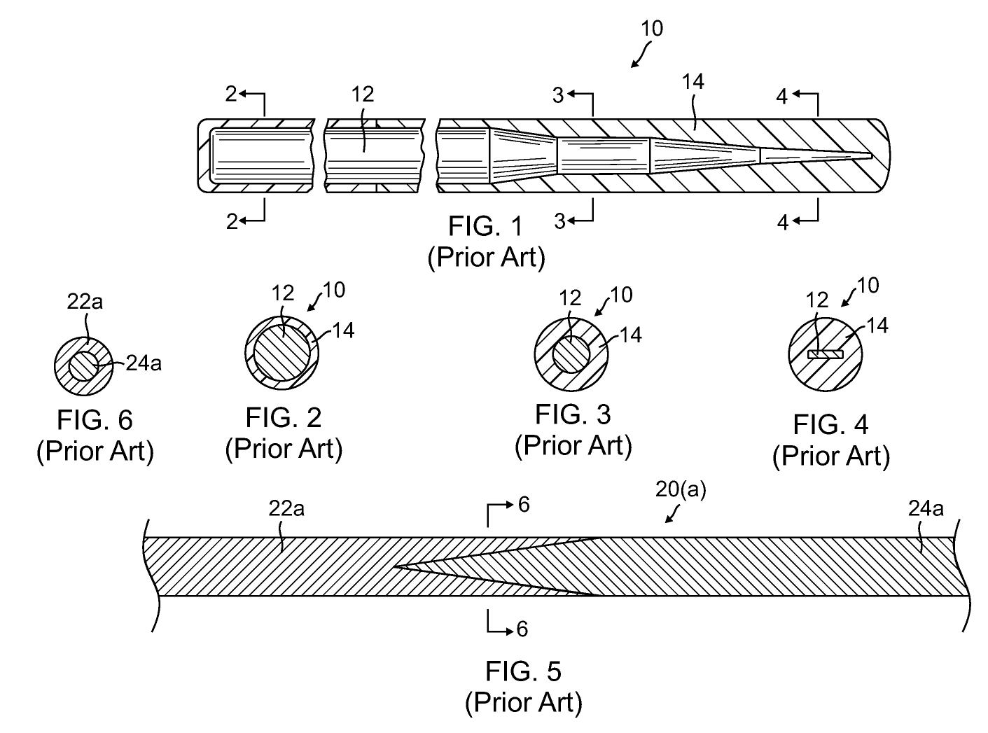

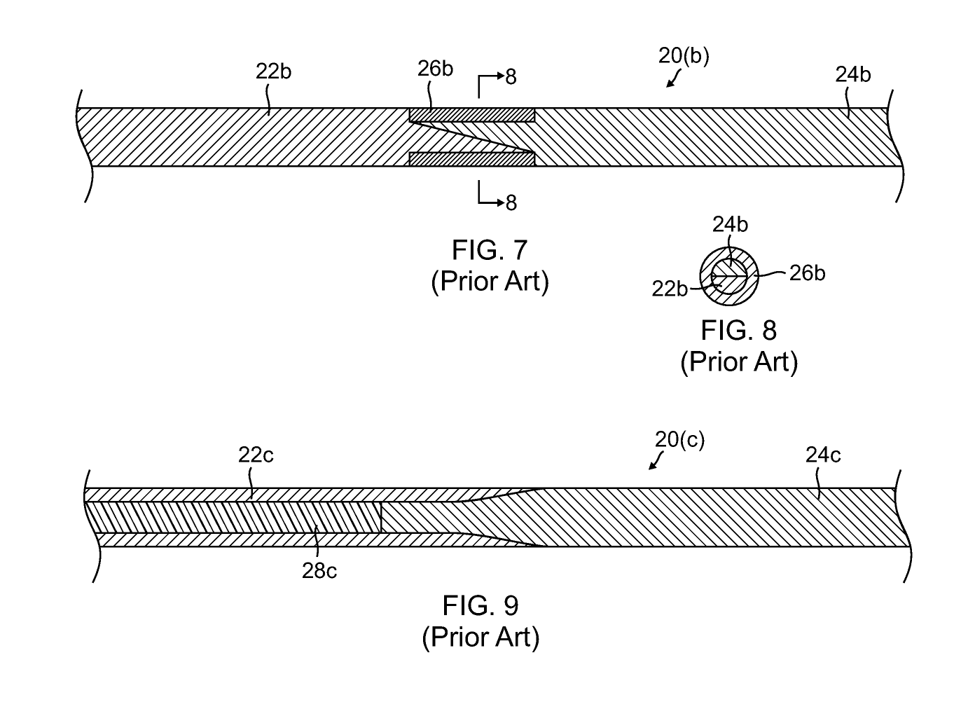

### DETAILED DESCRIPTION OF THE EMBODIMENTS

In conjunction with the figures, there is described herein a medical guidewire and a method for manufacturing a medical guidewire having features of an embodiment of the present invention. In some embodiments, the invention includes a method for forming a core for a guide wire of an embodiment according to the present invention.

In its final form, the guidewire may comprise an elongated solid core wire 112 and an outer jacket 114 made from a polymer with lubricious, or with hydrophilic or even with hydrophobic qualities, depending on the needs of the situation, The elongated solid core wire 112 includes a proximal section 116 of a constant diameter, and a distal section 118.

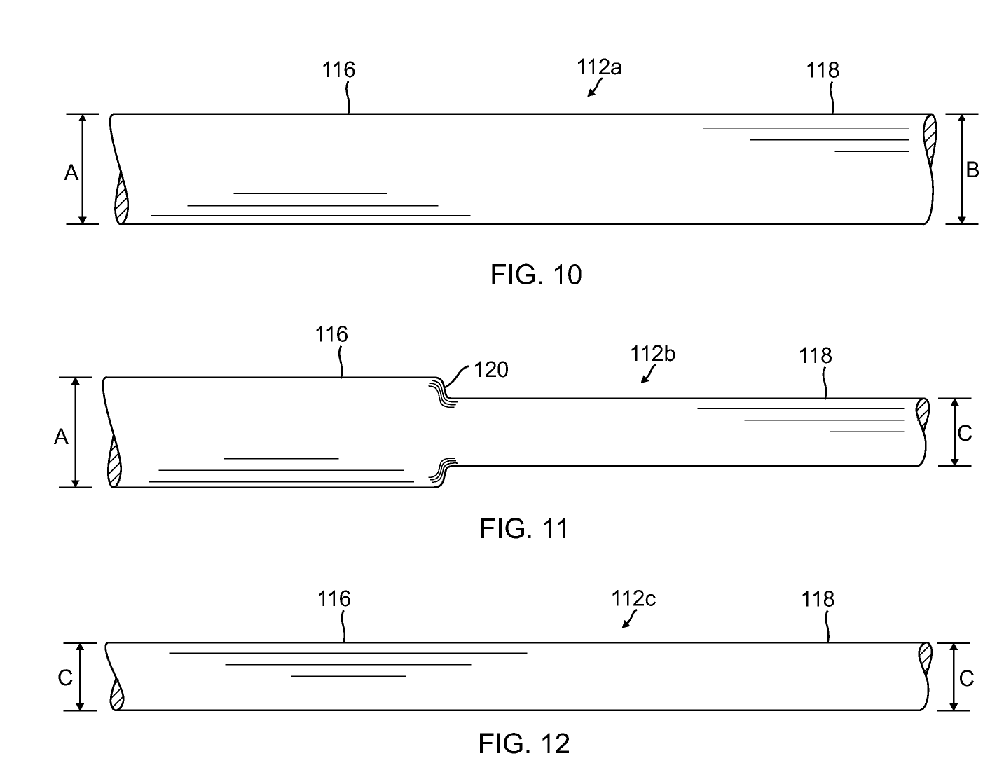

The core wire may preferably be made of a NiTi alloy. In some embodiments, the NiTi alloy useful for the present invention may be initiated by preparing an ingot which is melted and cast using a vacuum induction or vacuum arc melting process. The ingot is then forged, rolled and drawn into a wire. In some embodiments, exemplified in FIG. 10, the resulting core wire 112a may have a diameter of about 0.030 inches in diameter, and may have a nominal composition of about 55.0 weight percent Ni and an austenite transformation start (As) temperature of about 0 degree C. in the fully annealed state. In this form, the wire may exhibit superelastic properties at a body temperature of about 37 degree C., which are desirable in at least portions of a guidewire so that those portions do not permanently deform as they are extended through a tortuous anatomy.

Once the initial basic wire 112a has been thus prepared, a length of wire that is desired to possess linear elastic properties is identified and selected. With reference to FIGS. 11 to 14, this selected length is identified by the reference numeral 118 and is referred to herein as the distal portion of the wire. A portion of the wire that is not desired to possess linear elastic properties, hut to retain its superelastic properties, is identified by the numeral 116 and is referred to herein as the proximal portion. In some embodiments, the proximal portion 116 and the distal portion 118 are selected to be adjacent to each other, but this is not a limiting requirement of the invention. In fact, portions of the wire between the proximal portion 116 and the distal portion 118 may be selected for yet further and different treatment than that set forth herein below. In this initial condition, the wire is configured so that the proximal portion has a diameter “A,” and the distal portion may have a second diameter “B” as shown in FIG. 10. In some embodiments, the first diameter A is the same as the second diameter B, while in other embodiments these diameters may purposely differ and may have a gradual taper between them.

In either case, the following manufacturing steps may be performed. Cold work may be applied to the distal portion 118 of the wire, without applying cold work to the proximal portion 116 of the wire. By applying cold work to the distal portion 118, the diameter of the distal portion is given a third diameter “C” that is less than the second diameter “B”, as seen in FIG. 11. In some embodiments, the cold work may be applied by drawing the distal portion through a die and then removing it by reverse drawing. This overall process may further include removing the wire from the die without drawing the distal portion 118 back through the die, such as by using a multiple-piece die which can be opened to enable wire removal, In other embodiments, applying cold work to the distal portion may include methods selected from swaging, tensioning, rolling, stamping, and coining. In some embodiments, swaging may utilize a set of two or more revolving dies which radially deform the workpiece repeatedly as it passes between the dies Like wiredrawing, swaging can produce an essentially round cross-section of reduced diameter. However the resulting work hardening is typically non-uniform across its final cross-section due to the so-called “redundant work” caused by repeated re-ovalization as the revolving dies repeatedly strike the non-revolving workpiece (which may be in 60° increments, in some embodiments). The final distribution of cold work may be influenced by both feed rate and die strike rate, and likely also by the contact length of the die set. Hence, judicious selection of processing conditions is required to attain the desired level of cold work within the distal section of the Nitinol core wire before grinding to final size.

Regardless of initial straightness of a wire, it is typical for as-drawn wire to become curved as a result of passing through a wiredrawing die. This can be remedied by simultaneously applying heat and tension to induce stress relaxation within the as-drawn portion. This straightening method can be applied to the present invention, provided the time and temperature are not sufficient to restore original superelastic properties, which typically takes several minutes at about 500° C. A suitable combination of tension and heat may be determined through experimentation, with the goal of attaining suitable straightness for a drawn portion, which persists after producing the final guide wire core profile.

Once the wire is given satisfactory metallurgical properties by differential treatments such as those described, it will be appreciated that the wire may have a stepped shoulder 120 as exemplified by wire 112b seen in FIG. 11, where the distal portion 118 may have linear elastic properties, and the proximal portion 116 may retain the original superelastic properties inherent in the unworked nickel titanium alloy. It will be appreciated that the step 120 seen in FIG. 11 may have a steep stepped gradient, or a more gently sloping gradient, depending on the precise process by which cold work is applied to the distal portion 118.

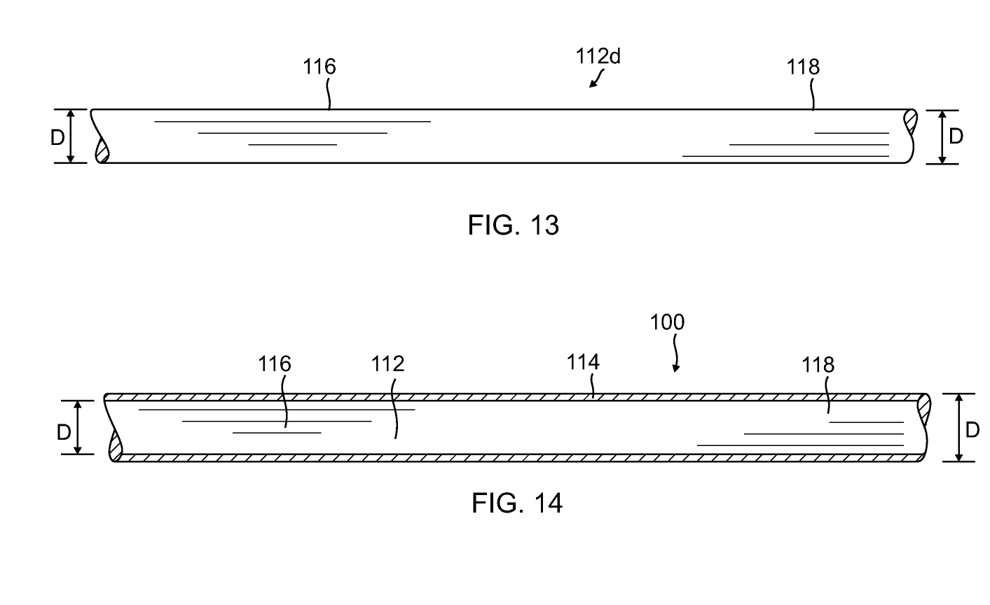

In a subsequent stage, the wire may then be subjected to a reducing process, in which the step 120, (i.e., the differential diameter between the proximal portion 116 and the distal portion 118) is removed. In this stage, the step 120 may be removed to impart the proximal portion 116 of the wire 112c to have a diameter “C” that is the same as the existing third diameter “C” of the distal portion 118, as seen in FIG. 12. Alternatively, the wire 112d may be further reduced so that both proximal and distal portions are reduced so that each has have a fourth diameter “D” that is smaller than diameter “C”, as seen in FIG. 13.

In some embodiments, the process of reducing the wire may be the known process of centerless grinding, which is a machining process that uses abrasive cutting to remove material from a workpiece. In some forms of centerless grinding, the workpiece is held between a workholding platform and two wheels rotating in the same direction at different speeds. One wheel, known as the regulating wheel, is on a fixed axis and rotates such that the force applied to the workpiece is directed downward, against the workholding platform. This wheel usually imparts rotation to the workpiece by having a higher linear speed than the other wheel. The other wheel, known as the grinding wheel, is movable. This wheel is positioned to apply lateral pressure to the workpiece, and usually has either a very rough or a rubber-bonded abrasive to grind away material from the workpiece. The speed of the two wheels relative to each other provides the rotating action and determines the rate at which material is removed from the workpiece by the grinding wheel. During operation the workpiece turns with the regulating wheel, with the same linear velocity at the point of contact and (ideally) no slipping. The grinding wheel turns faster, slipping past the surface of the workpiece at the point of contact and removing chips of material as it passes. In other embodiments of the invention, the reducing process may include chemical washes, or polishes.

Once these reducing steps as described above are performed, the wire 112c or 112d will have a uniform diameter “C” or “D” respectively throughout the proximal portion and distal portion. It will be appreciated however that, despite its uniform geometrical shape the wire will have differential metallurgical properties in the proximal and distal portions, and hence differential flexural and torsional stiffnesses and also deformation related properties.

Thus, once a uniform wire of desired diameter is produced according to the methodology set forth, the wire may be coated with a suitable lubricious polymer coating 114 as seen in FIG. 14. The wire thus produced does not have unnecessary joints between portions having different metallurgical properties, and neither does it have unnecessary diametric steps between different portions. This aspect eliminates focus points or stress raising points for kinking for fracture, and results in a strong and reliable core wire that has beneficial differential properties along its length that may affect torsional stiffness while allowing differential flexibility as desired for vascular insertion. By way of example, a guide wire core wire thus produced may provide non-superelastic metallurgical properties to its extreme distal end directly after centerless grinding, without need for subsequent deformation such as flattening to impart said properties, thus enabling a fully circular cross-section with its associated rotational bending uniformity which prevents the alternating buildup then release of stored elastic energy, known as “whipping”, when the guide wire is rotationally manipulated in tortuous anatomy.

As used herein, the terms proximal and distal do not necessarily reflect a proximal-most portion or a distal-most portion of a guidewire element. Rather, these terms are used to indicate the position of one portion in relation to another. Additional portions may be added to either end of a proximal or a distal portion and that are not subjected to the processes set forth herein.

Thus, the embodiments described provide an advantageous system and method for manufacturing a medical guidewire. The present invention may, of course, be carried out in other specific ways than those herein set forth without departing from the essential characteristics of the invention. The present embodiments are, therefore, to be considered in all respects as illustrative and not restrictive, while the scope of the invention is set forth in the claims that follow.

We claim:

1. A method of making a core metal element for a medical guidewire comprising:
providing a wire of nickel titanium alloy having a length that includes a proximal portion having a first diameter and a distal portion having a second diameter;
applying cold work to the distal portion and not applying cold work to the proximal portion, thereby imparting to the distal portion a third diameter that is smaller than the second diameter: and then
applying a reducing process to the wire whereby the proximal portion is reduced to have a fourth diameter that is less than the first diameter.

2. The method of claim 1, wherein providing a wire includes providing a wire with superelastic properties throughout the length.

3. The method of claim 1, wherein applying cold work to the distal portion includes applying sufficient cold work to render the distal portion to have linear elastic properties.

4. The method of claim 3, wherein, after applying cold work to the distal portion, the proximal portion retains superelastic properties.

5. The method of claim 4, wherein no welding process is applied to the wire over the length.

6. The method of claim 4, wherein no joint is inserted into the wire over the length.

7. The method of claim 1, wherein applying a reducing process to the guidewire includes applying centerless grinding.

8. The method of claim 1, wherein providing a wire includes providing a wire wherein the first diameter is the same as the second diameter.

9. The method of claim 1, wherein applying a reducing process to the wire includes reducing the proximal portion to have a fourth diameter that is the same as the third diameter.

10. The method of claim 1, wherein applying a reducing process to the wire includes reducing the distal portion to have a fifth diameter that is less than the third diameter.

11. The method of claim 1, wherein applying cold work to the distal portion includes drawing the distal portion through a die.

12. The method of claim 11, further including removing the guidewire from the die without drawing the distal portion back through the die.

13. The method of claim 1, wherein applying cold work to the distal portion includes applying cold work methods selected from: swaging, rolling, tensioning, stamping, and coining.

14. The method of claim 1, wherein providing a wire includes providing a wire wherein the proximal portion is adjacent the distal portion.

15. The method of claim 1, wherein providing a wire includes providing a wire wherein the proximal portion is adjacent a proximal end of the wire.

16. The method of claim 1, wherein providing a wire includes providing a wire wherein the distal portion is adjacent a distal end of the wire,

17. A medical guidewire comprising:
a solid metal core having a length and having a constant diameter over the length, wherein the length includes a proximal portion having pseudoelastic properties and a distal portion having linear elastic properties, and wherein the length of the core does not include a mechanical joint at any location between the proximal portion and the distal portion.

18. The guidewire of claim 17, wherein the proximal portion is formed from a nickel titanium alloy.

19. The guidewire of claim 17, wherein the distal portion includes metal to which the linear elastic properties have been imparted by a process of cold working.
